(19) United States
(12) Patent Application Publication
(10) Pub. No.: US 2012/0070419 A1
(43) Pub. Date: Mar. 22, 2012

(54) METHOD OF ALTERING THE DIFFERENTIATIVE STATE OF A CELL AND COMPOSITIONS THEREOF

(75) Inventor: Trudy Christiansen-Weber, Oceanside, CA (US)

(73) Assignee: INTERNATIONAL STEM CELL CORPORATION, Oceanside, CA (US)

(21) Appl. No.: 13/053,054

(22) Filed: Mar. 21, 2011

Related U.S. Application Data
(60) Provisional application No. 61/324,620, filed on Apr. 15, 2010.
(60) Provisional application No. 61/317,650, filed on Mar. 25, 2010.

(51) Int. Cl. A61K 35/12; C12N 5/071; C12Q 1/02; C12N 5/0775; C07K 19/00; C12N 15/85; C12N 5/0735

(52) U.S. Cl. 424/93.21; 435/455; 435/377; 435/320.1; 530/350; 424/93.7; 435/29

(57) ABSTRACT

The present invention provides a method of altering the differentiative state of cells utilizing innovative protein expression constructs encoding transcription factors. The methods and compositions described herein may be used to generate induced pluripotent stem (iPS) cells, as well as differentiate, transdifferentiate or dedifferentiate cells of various epigenetic status. The method includes introduction of a nucleic acid construct, or expression product thereof, into a cell, and culture of the cell under culture conditions that efficiently converts the cell into a pluripotent cell, enhances the retention of the pluripotent state or efficiently converts the cell into a cell of a cell lineage corresponding to endoderm, mesoderm or ectoderm.

### CROSS REFERENCE TO RELATED APPLICATION(S)

This application claims the benefit of priority under 35 U.S.C. §119(e) of U.S. Ser. No. 61/324,620, filed Apr. 15, 2010, and U.S. Ser. No. 61/317,650, filed Mar. 25, 2010, the entire content of which are incorporated herein by reference in their entirety.

### BACKGROUND OF THE INVENTION

1. Field of the Invention

The present invention relates generally to stem cells, and more specifically to a method and compositions for altering the differentiative state of a cell, and cells generated therefrom.

2. Background Information

During embryonic development, the tissues of the body are formed from three major cell populations: ectoderm, mesoderm and endoderm. These cell populations, also known as primary germ cell layers, are formed through a process known as gastrulation. Following gastrulation, each primary germ cell layer generates a specific set of cell populations and tissues. For example, mesoderm gives rise to blood cells, endothelial cells, cardiac and skeletal muscle, and adipocytes; endoderm generates liver, pancreas and lung; and ectoderm gives rise to the nervous system, skin and adrenal tissues.

Human embryonic stem (hES) cells are pluripotent cells that can differentiate into a large array of cell types. When injected into immune-deficient mice, embryonic stem cells form differentiated tumors (teratomas). However, embryonic stem cells that are induced in vitro to form embryoid bodies (EBs) provide a source of embryonic stem cell lines that are amenable to differentiation into multiple cell types characteristic of several tissues under certain growth conditions. Various methods are known for the reprogramming of both mouse and human somatic cells to pluripotent stem cells, termed induced pluripotent stem (iPS) cells. Following reprogramming, differentiation of the iPS cells to a specific tissue type may be initiated.

Various types of stem cells and their progeny are amenable to reprogramming, differentiation, dedifferentiation and transdifferentiation and are important sources of normal human cells for therapeutic transplantation, such as hepatocytes, and for drug testing and development. Such goals require sufficient cells which are differentiated into tissue types suitable for a patient's needs or the appropriate pharmacological test. Associated with this is a need for an efficient and reliable method of altering the differentiative state of a cell, such as by reprogramming, differentiating, dedifferentiating and transdifferentiating cells. Provided herein is such a method, capable of producing highly enriched populations of cells of identical differentiative status.

### SUMMARY OF THE INVENTION

The present invention is based on the seminal discovery of an innovative method and nucleic acid constructs for altering the differentiative state of a cell, such as reprogramming cells to generate induced pluripotent stem (iPS) cells, as well as differentiating, transdifferentiating or dedifferentiating cells of various differentiative status.

As such, the present invention provides a method of reprogramming, differentiating, transdifferentiating or dedifferentiating a target or recipient cell into a cell of a different cell type or into a pluripotent or less differentiated cell. The method includes introduction of a nucleic acid construct, or the expression product thereof, into the cell and culture under culture conditions that convert the cell into a pluripotent cell or into a cell of a cell lineage corresponding to endoderm, mesoderm or ectoderm.

In various aspects the method utilizes a nucleic acid construct encoding a cassette including in operable linkage: i) at least one protein tag; ii) a protein transduction domain; iii) a fusion domain; iv) a nuclear localization signal; and v) at least one transcription factor. Accordingly, the present invention further provides a nucleic acid construct.

In one embodiment, the transcription factor of the construct is a nuclear reprogramming factor or transcription factor involved in differentiation or transdifferentiation. In various aspects, the transcription factor is encoded by a SOX family gene, a KLF family gene, a MYC family gene, SALL4, OCT4, NANOG, LIN28, or a combination thereof, such as OCT4, SOX2, KLF4, NANOG, or c-MYC. In related aspects, the transcription factor is encoded by a gene including OCT4, NANOG, SOX2, SOX17, HNF4, GATA4, HHEX, CEBPβ, CEBPδ, PRDM16, MYOD1, NKX2.5, MEF2c, MYOCARDIN, RUNX2, PDX, NGN, SALL4 or SOX9, or combination thereof, such as Oct4, NANOG, Sox2, Sox9, Sax 17, HNF4α2, HNF4α4, HNF4α7, HNF4α8, HNF4γ, GATA4, Hhex, CEBPβ, CEBPδ, PRDM16, MyoD1, NKX2.5, Mef2c, Myocardin, Runx2-I, Pdx1, Ngn3, Sall4 or Runx2-II.

In various aspects, the nucleic acid construct encodes at least one protein tag, such as poly(His), haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO), ubiquitin (Ub), or combination thereof. In one embodiment, the nucleic acid construct encodes at least two protein tags, such as an affinity tag and an epitope tag. In a related embodiment, the at least two protein tags include a poly(His) tag and a haemagglutinin (HA) epitope tag.

In various aspects, the nucleic acid construct encodes a fusion domain. In one embodiment, the fusion domain includes influenza hemagglutinin fusion peptide or fragment thereof.

In a related aspect, the nucleic acid construct encodes a protein transduction domain. In one embodiment, the protein transduction domain includes a TAT protein, VP22 protein, Drosophila Antennapedia (Antp) homeotic transcription factor, or fragments thereof.

In one aspect, the nucleic acid construct encodes in operable linkage, a transcription factor, poly(His) tag, haemagglutinin (HA) tag, TAT protein, influenza hemagglutinin fusion peptide, and nuclear localization signal (NLS). In various embodiments, each element is separated by between 1 and 10 amino acids. In an exemplary embodiment, each element is spaced by at least 2 amino acids, such as glycine, to allow for free rotation of each element independent of each individual element.

In various aspects, the target or recipient cell may be undifferentiated, partially differentiated, or fully differentiated before the nucleic acid construct, or expression product thereof, is introduced into the cell. In related aspects, the target or recipient cell may be an embryonic stem (ES) cell, a pluripotent stem (PS) cell, an induced pluripotent stem (iPS) cell, a parthenogenetic stem cell, a multipotent stem cell, a bipotent stem cell, a mesenchymal stem cell, an endodermal stem cell, an ectodermal stem cell, a somatic stem cell or a somatic cell.

In another aspect, the nucleic acid construct encodes at least one additional cassette, each including in operable linkage: i) at least one protein tag; ii) a protein transduction domain; iii) a fusion domain; iv) a nuclear localization signal; and v) at least one transcription factor. As such, multiple transcription factors may be expressed from a single nucleic acid construct. Similarly, to allow for expression of more than one transcription factor, at least one additional nucleic acid construct, or the expression product thereof, as described herein may be introduced into the cell.

In another aspect, the present invention provides an expression vector including the nucleic acid construct of the invention.

In another aspect, the present invention provides an isolated protein encoded by the nucleic acid construct of the present invention. Expression of the nucleic acid construct generates a chimeric protein including a transcription factor fused to at least one protein tag, a protein transduction domain, a fusion domain, and an NLS, the individual elements being in any order.

In another aspect, the present invention provides a method of generating induced pluripotent stem cells (iPS) with a higher degree of efficiency than the original unmodified transcription factors. The method includes reprogramming a somatic cell into an iPS cell using multiple chimeric proteins including a transcription factor fused to at least one protein tag, a protein transduction domain, a fusion domain, and an NLS, the individual elements being in any order.

In another aspect, the present invention provides a method of enhancing retention of the pluripotency of pluripotent stem cells, including embryonic stem cells or parthenogenetic stem cells. The method includes reprogramming differentiated cells present in said pluripotent stem cell cultures into a pluripotent stem cells using multiple chimeric proteins including a transcription factor fused to at least one protein tag, a protein transduction domain, a fusion domain, and an NLS, the individual elements being in any order.

In another aspect, the present invention provides a method of directing the differentiation of multipotent stem cells or pluripotent stem cells (including embryonic stem cells, parthenogenetic stem cells or induced pluripotent stem cells) to a targeted fate. The method includes reprogramming unwanted differentiated cells generated during embryoid body formation using multiple chimeric proteins including a transcription factor fused to at least one protein tag, a protein transduction domain, a fusion domain, and an NLS, the individual elements being in any order.

In yet another aspect, the present invention provides a method of treating a subject utilizing cells derived from the methods described herein, wherein iPS cells are initially generated and subsequently differentiated into a specific cell type. The method includes obtaining a somatic cell from a subject; reprogramming the somatic cell into an induced pluripotent stem (iPS) cell using the methods of the invention; culturing the induced pluripotent stem (iPS) cell ex vivo to differentiate the cell into a desired cell type suitable for treating a condition; and introducing into the subject the differentiated cell, thereby treating the condition.

In a related aspect, the present invention provides a method of treating a subject utilizing cells derived from the methods described herein wherein an iPS cell is not generated initially and subsequently differentiated. The method includes contacting a cell with the nucleic acid construct of the invention, or the expression product thereof, culturing the cell to differentiate, transdifferentiate or dedifferentiate the cell into a desired cell type suitable for treating a condition; and introducing the cultured cell into the subject, thereby treating the condition.

### BRIEF DESCRIPTION OF THE DRAWINGS

FIG. 1 is a schematic of the two-stage PCR reaction for creation of recombinant proteins including a carboxy terminal domain of one embodiment of the present invention which includes a first protein tag (an epitope haemagglutinin tag (HA)), a transduction domain (TAT), a second protein tag (a poly(His) tag), a fusion domain (influenza hemagglutinin fusion peptide), and a nuclear localization sequence (NLS to target the protein to the nucleus), the complete domain referred to as HATHFUN. Partial HATHFUN primer sequences are shown as SEQ ID NO: 43 (1st PCR Reaction, 5′ to 3′) and SEQ ID NO: 44 (2nd PCR Reaction, 5′ to 3′).

### DETAILED DESCRIPTION OF THE INVENTION

The present invention provides a method of altering the differentiative state of cells utilizing innovative protein expression constructs encoding transcription factors. The method and compositions described herein may be used to generate induced pluripotent stem (iPS) cells, as well as differentiate, transdifferentiate or dedifferentiate cells of various epigenetic status. The method includes introduction of a nucleic acid construct, or the expression product thereof into a cell, and culture of the cell under culture conditions that convert the cell into a pluripotent cell or into a cell of a cell lineage corresponding to endoderm, mesoderm or ectoderm.

Before the present composition, methods, and culturing methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

Cellular differentiation is the process by which a less specialized cell becomes a more specialized cell type, often accompanied by dramatic changes in cellular characteristics, such as cell size, shape, membrane potential, metabolic activity, and responsiveness to signals. These changes are largely due to highly-controlled modifications in gene expression. Cell differentiation is thus a transition of a cell from one cell type to another and typically involves a switch from one pattern of gene expression to another.

As such, the present invention provides a method of altering the differentiative state of cells utilizing innovative protein expression constructs encoding transcription factors, the expression or introduction of the encoded protein within a cell altering the differentiative state of the cell. The protein expression constructs of the present invention provide a method of reprogramming, differentiating, transdifferentiating or dedifferentiating a target or recipient cell into a cell of a different cell type or into a pluripotent or less differentiated cell. The method includes introduction of a nucleic acid construct into the cell, or the expression product thereof, e.g., a chimeric protein, and culture under culture conditions that convert the cell into a pluripotent cell or into a cell of a cell lineage corresponding to endoderm, mesoderm or ectoderm.

As discussed herein, various aspects of the present invention relate to in vitro methodology that results in conversion of cells of one differentiative state to that of another. Such methods encompass the application of culture and growth factor conditions in a defined and temporally specified fashion. In various embodiments, the method of the present invention generates a cell population in which 10%, 20%, 30%, 40%, 50%, 60%, 70%, 80%, 90% or greater of the cells have an altered differentiative state. For example, the invention provides cell populations in which about 50-99%, 60-99%, 70-99%, 75-99%, 80-99%, 85-99%, 90-99% or 95-99% of the cells in culture are of a similar differentiative state. Further enrichment of the cell population for a particular cell type can be achieved by isolation and/or purification of altered cells from other cells in the population, for example by using reagents known in the art that specifically bind a particular cell type.

Culture of the cells in which a nucleic acid construct has been introduced may be performed in the presence of additional maturation or growth factors useful for generating a specific cell type. For example, differentiation from human embryonic stem cells may be assisted by providing to the stem cell culture a growth factor of the TGFβ superfamily, such as Nodal/Activin proteins or BMP subgroup proteins. Additional growth factors, such as Wnt3a and other Wnt family members are useful in the culture medium. While any additional agent or growth factor may be added to the culture medium, in exemplary aspects, the maturation or growth factor is Nodal, bFGF, Activin A, Activin B, BMP4, Wnt3a, Oncostatin M, bile salt and combinations thereof.

In various aspects the method utilizes a nucleic acid construct encoding a cassette including in operable linkage: i) at least one protein tag; ii) a protein transduction domain; iii) a fusion domain; iv) an NLS; and v) at least one transcription factor. Accordingly, the present invention provides a nucleic acid construct.

In addition, the present invention provides the expression product of the nucleic acid construct, a chimeric protein. The chimeric protein includes i) at least one protein tag; ii) a protein transduction domain; iii) a fusion domain; iv) an NLS; and v) at least one transcription factor.

As used herein, the term “operatively linked” or “operable linkage” means that two or more molecules are positioned with respect to each other such that they act as a single unit and effect a function attributable to one or both molecules or a combination thereof. For example, a polynucleotide encoding a gene can be operatively linked to a transcriptional or translational regulatory element, in which case the element confers its regulatory effect on the polynucleotide similar to the way in which the regulatory element would effect a polynucleotide sequence with which it normally is associated with in a cell.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

The term “nucleic acid construct” or “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked, such as a gene encoding a transcription factor, and one or more protein tags, functional domains and the like.

In various aspects, the nucleic acid construct encodes at least one protein tag. A variety of protein tags are known in the art, such as epitope tags, affinity tags, solubility enhancing tags, and the like. Affinity tags are the most commonly used tag for aiding in protein purification while epitope tags aid in the identification of proteins. One of skill in the art would understand that some tags may be useful as more than one type of tag. For example, a poly(His) tag may serve as an epitope tag as well as an affinity tag. Examples of various tags that may be used with the present invention include poly(His), haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO), ubiquitin (Ub), or any combinations thereof. In one embodiment, the nucleic acid construct encodes at least two protein tags, such as an affinity tag and an epitope tag. In a related embodiment, at least two protein tags include a poly(His) tag and a haemagglutinin (HA) epitope tag.

As used herein, a poly(His) tag is an amino acid motif that includes at least five histidine residues, typically at the N-terminus or C-terminus of a protein. For example, a poly(His) tag may include about 5, 6, 7, 8, 9, 10 or more consecutive histidine residues.

The nucleic acid construct of the present invention may be introduced into a cell to be altered thus allowing expression of the chimeric protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™.

Alternatively, the chimeric protein may be generated by expression in an appropriate bacterial expression system including bacterial cells transformed with an expression vector including the nucleic acid construct. The chimeric protein is then contacted with the cell to be altered in culture, the chimeric protein gaining entry into the cell via a protein transduction domain. Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be incorporated into the chimeric protein of the present invention to successfully transport the protein into a cell. In exemplary embodiment, the nucleic acid construct encodes a protein transduction domain which is a TAT protein, VP22 protein, Drosophila Antennapedia (Antp) homeotic transcription factor, or fragments thereof. In an exemplary embodiment, the nucleic acid construct encodes a protein transduction domain having the following amino acid sequence:

| | |
|---|---|
| YGRKKRRQRRR. | (SEQ ID NO: 1) |

To assist in the efficiency of the protein transduction domain in facilitating entry of the protein into a cell, the nucleic acid construct may additionally encode a fusion domain. A number of synthetic and naturally occurring fusion domains are known in the art which may be used in the present invention. In an exemplary embodiment, the fusion domain includes influenza hemagglutinin fusion peptide or fragment thereof. In an exemplary embodiment, the nucleic acid construct encodes a fusion domain having the following amino acid sequence:

| | |
|---|---|
| GLFGAIAGFIENGWEGMIDG. | (SEQ ID NO: 2) |

The nucleic acid construct of the present invention further includes a nuclear localization sequence (NLS) for directing the chimeric protein to the nucleus of the cell. Any NLS sequence known in the art may be used in the present invention. In an exemplary embodiment, the nucleic acid construct encodes a NLS having the following amino acid sequence:

| | |
|---|---|
| KKKRKV. | (SEQ ID NO: 3) |

The protein tag, protein transduction domain, fusion domain, and NLS may be encoded in any position of the nucleic acid construct and in any order, so long as the individual elements and the transcription factor are functional upon generation of the chimeric protein. Typically, the protein tag, protein transduction domain, fusion domain, and NLS are encoded downstream of the transcription factor such that they are arranged at the carboxy-terminus of the chimeric protein.

In one embodiment, the nucleic acid construct encodes in operable linkage, a transcription factor, poly(His) tag, haemagglutinin (HA) tag, TAT protein or fragment thereof, influenza hemagglutinin fusion peptide or fragment thereof, and an NLS. In various embodiments, each element is separated by between 1 and 10 amino acids. In an exemplary embodiment, each element is spaced by 2 amino acids, such as glycines, to allow for free rotation of each element independent of each other element. In an exemplary embodiment, the chimeric protein encoded by the nucleic acid construct includes a transcription factor and a domain including a poly(His) tag, haemagglutinin (HA) tag, TAT protein or fragment thereof, influenza hemagglutinin fusion peptide or fragment thereof, and NLS, the domain having the following amino acid sequence:

(SEQ ID NO: 4)
YPYDVPDYAGGKKKRKVGGYGRKKRRQRRRGGHHHHHHGGGLFGAIAGFI
ENGWEGMIDG.

In various aspects, the nucleic acid construct encoding an expression cassette of the present invention further includes one or more promoters. As used herein, a promoter is intended mean a polynucleotide sequence capable of facilitating transcription of genes in operable linkage with the promoter. Several types of promoters are well known in the art and suitable for use with the present invention, for example constitutive promoters that allows for unregulated expression in mammalian cells, such as the cytomegalovirus (CMV) promoter.

Alternatively, the nucleic acid may include one or more inducible promoters. An inducible promoter is a promoter that, in the absence of an inducer (such as a chemical and/or biological agent), does not direct expression, or directs low levels of expression of an operably linked gene (including cDNA), and, in response to an inducer, its ability to direct expression is enhanced. Exemplary inducible promoters include, for example, promoters that respond to heavy metals, to thermal shocks, to hormones, and those that respond to chemical agents, such as glucose, lactose, galactose or antibiotic.

Advances in cloning technology allow generation of the nucleic acid constructs and vectors of the present invention. For example, Gateway® cloning technology, developed by Invitrogen Inc., enables the orienting and insertion of multiple polynucleotide fragments into a target vector.

In various aspects of the present invention, genes that encode transcription factors, known as nuclear reprogramming factors, capable of inducing pluripotency are utilized to reprogram differentiated or incompletely differentiated cells to a phenotype that is more primitive than that of the initial cell, such as the phenotype of an iPS cell. Such factors are capable of generating an iPS cell from a differentiated cell, such as a somatic cell upon expression of one or more such factors within the host cell via the nucleic acid construct of the present invention, or by introducing or contacting a host cell with a chimeric protein of the present invention including a nuclear reprogramming factor. As used herein, a gene that induces pluripotency or a nuclear reprogramming factor is intended to refer to a gene or factor that is associated with pluripotency and capable of generating a less differentiated cell, such as an iPS cell from a somatic cell. The expression of a pluripotency gene is typically restricted to pluripotent stem cells, and is crucial for the functional identity of pluripotent stem cells.

As used herein, a “pluripotent cell” refers to a cell that can be maintained in vitro for prolonged, theoretically indefinite period of time in an undifferentiated state, that can give rise to different differentiated tissue types, i.e., ectoderm, mesoderm, and endoderm. The pluripotent state of the cells is preferably maintained by culturing inner cell mass or cells derived from the inner cell mass of an embryo produced by androgenetic or gynogenetic methods under appropriate conditions, for example, by culturing on a fibroblast feeder layer or another feeder layer or culture that includes leukemia inhibitory factor (LIF). The pluripotent state of such cultured cells can be confirmed by various methods, e.g., (i) confirming the expression of markers characteristic of pluripotent cells; (ii) production of chimeric animals that contain cells that express the genotype of the pluripotent cells; (iii) injection of cells into animals, e.g., SCID mice, with the production of different differentiated cell types in vivo; and (iv) observation of the differentiation of the cells (e.g., when cultured in the absence of feeder layer or LIF) into embryoid bodies and other differentiated cell types in vitro.

Several genes have been found to be associated with pluripotency and suitable for use with the present invention. Such genes are known in the art and include, by way of example, SOX family genes (SOX1, SOX2, SOX3, SOX15, SOX18), KLF family genes (KLF1, KLF2, KLF4, KLF5), MYC family genes (C-MYC, L-MYC, N-MYC), SALL4, OCT4, NANOG, LIN28, and combinations thereof. While in some instances, use of only one gene to induce pluripotency may be possible, in general, expression of more than one gene is required to induce pluripotency. For example, two, three, four or more genes may be utilized. In an illustrative aspect, one or more genes encoding the following nuclear reprogramming factors are utilized: OCT4, SOX2, KLF4, NANOG, and c-MYC.

As used herein, reprogramming, is intended to refer to a process that alters or reverses the differentiation status of a somatic cell that is either partially or terminally differentiated. Reprogramming of a somatic cell may be a partial or complete reversion of the differentiation status of the somatic cell. In an exemplary aspect, reprogramming is complete wherein a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

Somatic cells that may be reprogrammed may be primary cells or immortalized cells. Such cells may be primary cells (non-immortalized cells), such as those freshly isolated from an animal, or may be derived from a cell line (immortalized cells). In an exemplary aspect, the somatic cells are mammalian cells, such as, for example, human cells or mouse cells. They may be obtained by well-known methods, from different organs, such as, but not limited to skin, lung, pancreas, liver, stomach, intestine, heart, reproductive organs, bladder, kidney, urethra and other urinary organs, or generally from any organ or tissue containing living somatic cells, or from blood cells. Mammalian somatic cells useful in the present invention include, by way of example, adult stem cells, sertoli cells, endothelial cells, granulosa epithelial cells (including retinal pigment epithelial cells), neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, adipocytes (brown and white), cardiac muscle cells, other known muscle cells, and generally any live somatic cells. In particular embodiments, fibroblasts are used. The term somatic cell, as used herein, is also intended to include adult stem cells. An adult stem cell is a cell that is capable of giving rise to all or several cell types of a particular tissue. Exemplary adult stem cells include hematopoietic stem cells, neural stem cells, and mesenchymal stem cells.

Thus, the invention further provides iPS cells produced using the methods described herein, as well as populations of such cells. The reprogrammed cells of the present invention, capable of differentiation into a variety of cell types, have a variety of applications and therapeutic uses. The basic properties of stem cells, the capability to infinitely self-renew and the ability to differentiate into every cell type in the body make them ideal for therapeutic uses.

In addition the generating iPS cells, the method and compositions of the present invention may be used to generate a wide variety of additional cell types with differentiation, transdifferentiation and dedifferentiation. In various aspects, other genes encoding transcription factors useful for reprogramming, differentiating, dedifferentiating, or transdifferentiating a cell include OCT4, NANOG, SOX2, SOX17, HNF4, GATA4, HHEX, CEBPβ, CEBPδ, PRDM16, MYOD1, NKX2.5, MEF2c, MYOCARDIN, RUNX2, PDX, NGN, SALL4 or SOX9, or combination thereof. The transcription factors encoded include Oct4, NANOG, Sox2, Sox9, Sox 17, HNF4α2, HNF4α4, HNF4α7, HNF4α8, HNF4γ, GATA4, Hhex, CEBPβ, CEBPδ, PRDM16, MyoD1, Nkx2.5, Mef2c, Myocardin, Runx2-I, Pdx1, Ngn3, Sall4 or Runx2-II. For example, differentiation of mesoderm or fibroblasts to adipocytes, chondrocytes, osteocytes and myocytes may be performed using chimeric proteins including the following transcription factors: CEBPβ/CEBPδ (adipocytes), Sox9 (chondrocytes), Runx2 (osteocytes) and MyoD1 (myocytes). Additional genes known as reprogramming factors suitable for use with the present invention are disclosed in U.S. patent application Ser. No. 10/997,146 and U.S. patent application Ser. No. 12/289,873, incorporated herein by reference.

All of these genes commonly exist in mammals, including human, and thus homologues from any mammals may be used in the present invention, such as genes derived from mammals including, but not limited to mouse, rat, bovine, ovine, horse, and ape. Further, in addition to wild-type gene products, mutant gene products including substitution, insertion, and/or deletion of several (e.g., 1 to 10, 1 to 6, 1 to 4, 1 to 3, and 1 or 2) amino acids and having similar function to that of the wild-type gene products can also be used. Furthermore, the combinations of factors are not limited to the use of wild-type genes or gene products. For example, Oct4 chimeras or other Oct4 variants can be used instead of wild-type Oct4.

The present invention is not limited to any particular combination of transcription or reprogramming factors useful for reprogramming, differentiating, dedifferentiating, or transdifferentiating a cell. As discussed herein a transcription or reprogramming factor may comprise one or more gene products. The transcription or reprogramming factor may also comprise a combination of gene products as discussed herein. Each factor may be used alone or in combination with other factors as disclosed herein. Further, transcription or reprogramming factors of the present invention can be identified by screening methods, for example, as discussed in U.S. patent application Ser. No. 10/997,146, incorporated herein by reference.

Use of a factors in combination with additional agents that facilitate reprogramming, differentiating, dedifferentiating, or transdifferentiating a cell is also envisioned. For example, such agents may include those that upregulate expression or activity of an endogenous nuclear reprogramming gene to increase the induction efficiency as compared to use of a reprogramming factor alone. In various embodiments, induction efficiency may be increased by 10, 20, 30, 40, 50, 60, 70, 80, 90, 100, 150, 200, 300, 400 or even 500% as compared as compared to induction without the use of additional agents. For example, induction efficiency may be as high as 1, 2, 3, 4, 5, 6, 7, 8, 9, 10, 15, 20, 25 or 50% (e.g., percent of induced cells as compared with total number of starting somatic cells).

During the induction process, the somatic cell may be contacted with the nuclear reprogramming factor simultaneously or before the cell is contact with one or more additional agents. In various embodiments, the somatic cell is contacted with an additional agent about 1, 2, 3, 4, 5, 7, 8, 9, 10, 11, 12, 13, 14 or more days after induction of the cell is begun.

As used herein, the terms “polypeptide”, “peptide”, or “protein” are used interchangeably to designate a linear series of amino acid residues connected one to the other by peptide bonds between the alpha-amino and carboxy groups of adjacent residues.

While the domains defining the expression product of the nucleic acid construct of the invention may be defined by motif sequences, one skilled in the art would understand that peptides that have similar sequences may have similar biological functions. Therefore, peptides having substantially the same sequence or having a sequence that is substantially identical or similar to a domain or factor disclosed herein may be utilized. As used herein, the term “substantially the same sequence” includes a peptide including a sequence that has at least 80%, 85%, 90%, 95%, 96%, 97%, 98% or greater sequence identity with the sequences defining domains or factors described herein and which have substantially the same activity or function.

A further indication that two polypeptides are substantially identical is that one polypeptide is immunologically cross reactive with that of the second. Further, two polypeptides are considered substantially identical where the two peptides differ only by conservative substitutions.

The term “conservative substitution” is used in reference to proteins or peptides to reflect amino acid substitutions that do not substantially alter the activity (for example, antimicrobial activity) of the molecule. Typically conservative amino acid substitutions involve substitution of one amino acid for another amino acid with similar chemical properties (for example, charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K) 5) Isoleucine (I), Leucine (L), Methionine (M), Valine (V); and 6) Phenylalanine (F), Tyrosine (Y), and Tryptophan (W).

The term “amino acid” is used in its broadest sense to include naturally occurring amino acids as well as non-naturally occurring amino acids including amino acid analogs. In view of this broad definition, one skilled in the art would know that reference herein to an amino acid includes, for example, naturally occurring proteogenic (L)-amino acids, (D)-amino acids, chemically modified amino acids such as amino acid analogs, naturally occurring non-proteogenic amino acids such as norleucine, and chemically synthesized compounds having properties known in the art to be characteristic of an amino acid. As used herein, the term “proteogenic” indicates that the amino acid can be incorporated into a protein in a cell through a metabolic pathway.

The terms “identical” or percent “identity” in the context of two polynucleotide or polypeptide sequences, refer to two or more sequences or subsequences that are the same or have a specified percentage of amino acid residues that are the same, when compared and aligned for maximum correspondence, as measured using a sequence comparison algorithm or by visual inspection.

The phrase “substantially identical,” in the context of two polynucleotides or polypeptides, refers to two or more sequences or subsequences that have at least 80%, 85%, 90%, 95%, 96%, 97%, 98% or greater nucleotide or amino acid residue identity, when compared and aligned for maximum correspondence, as measured using a sequence comparison algorithm or by visual inspection.

As is generally known in the art, optimal alignment of sequences for comparison can be conducted, for example, by the local homology algorithm of Smith & Waterman ((1981) Adv Appl Math 2:482), by the homology alignment algorithm of Needleman & Wunsch ((1970) J Mol Biol 48:443), by the search for similarity method of Pearson & Lipman ((1988) Proc Natl Acad Sci USA 85:2444), by computerized implementations of these algorithms, by visual inspection, or other effective methods.

The invention further provides differentiated, transdifferentiated or dedifferentiated cells produced using the methods described herein, as well as populations of such cells. The cells of the present invention have a variety of applications and therapeutic uses.

Gastrulation is a critical stage in early human development during which the three primary germ layers are first specified and organized. As used herein, “ectoderm” is tissue responsible for the eventual formation of the outer coverings of the body (epidermis) and the entire nervous system. It emerges first and forms from the outermost of the germ layers. As used herein, “mesoderm” differentiates to give rise to heart, blood, bone, skeletal muscle and other connective tissues. Various mesodermal cells retain the capacity to differentiate in diverse directions, for example, some cells in the bone marrow (mesoderm) may differentiate to liver (endoderm). As used herein, “endoderm” refers to both “definitive endoderm” and primitive endoderm”. Definitive endoderm typically refers to the germ layer that is responsible for formation of the entire gut tube including the esophagus, stomach and small and large intestines, and the organs which derive from the gut tube such as the lungs, liver, thymus, parathyroid and thyroid glands, gall bladder and pancreas. A distinction may be made between the definitive endoderm and the completely separate lineage of cells termed primitive endoderm. The “primitive endoderm” is primarily responsible for formation of extra-embryonic tissues, mainly the parietal and visceral endoderm portions of the placental yolk sac and the extracellular matrix material of Reichert's membrane.

In accordance with certain embodiments, endoderm cells are produced. These cells may be mammalian cells, such as human cells. In some embodiments of the present invention, definitive endoderm cells express or fail to significantly express certain markers. In one non-limiting aspect, one or more markers selected from SOX17, CXCR4, MIXL1, GATA4, HNF3b, GSC, FGF17, VWF, CALCR, FOXQ1, CMKOR1, CRIP1, FoxA2 and/or Shh. In another embodiment, one or more markers selected from OCT4, alpha-fetoprotein (AFP), Thrombomodulin (TM), SPARC, SOX1 and SOX7 are not significantly expressed in the definitive endoderm cells. In another embodiment, the definitive endoderm cells do not express E-caherin and/or Oct4.

In some embodiments, the cells are further treated to form cells of the gastrointestinal tract, respiratory tract, or endocrine system. For example, the endodermal cells may be differentiated into cells of the organs of the gastrointestinal system, respiratory tract, or endocrine system. In particular aspects, the cells are further treated to form liver cells or pancreas cells. In some embodiments of the invention, hepatocyte progenitors, that start expressing AFP (day 7 or day 8 of differentiation) may be used in transplantation.

In other embodiments, mesoderm cells are produced. These cells may be further treated to form any cell derived from a mesoderm lineage. In some embodiments, mesoderm cells may be differentiated by methods known in the art into bone cells, muscle cells, connective tissue, or blood cells.

In other embodiments, ectoderm cells are produced. These cells may be further treated to form any cell derived from a ectoderm lineage. In some embodiments, ectoderm cells may be differentiated by methods known in the art into cells of the nervous system or skin.

In accordance with other embodiments of the present invention, methods of producing hepatocytes from pluripotent cells are described. In one embodiment, iPS cells are derived from somatic cells using the methods described herein. In another embodiment, pluripotent stem cells are stem cells. Stem cells used in these methods can include, but are not limited to, embryonic stem (ES) cells. In one embodiment, hES cells are used to produce hepatocytes.

The cell cultures and compositions comprising endoderm, mesoderm or ectoderm cells that are described herein can be produced from pluripotent cells, such as parthenogenetic stem cells (pSC), iPS cells or embryonic stem cells. iPS cells may be generated as described herein. As used herein, “embryonic” refers to a range of developmental stages of an organism beginning with a single zygote and ending with a multicellular structure that no longer comprises pluripotent or totipotent cells other than developed gametic cells. In addition to embryos derived by gamete fusion, the term “embryonic” refers to embryos derived by somatic cell nuclear transfer. As used herein, “parthenogenetic stem cells (pSC)” refers to pluripotent stem cells derived through the process of parthenogenesis. Parthenogenesis results in “parthenogenetic” embryos (or a “pathenote”) formed from activated unfertilized oocytes. A preferred method for deriving endoderm, mesoderm or ectoderm cells utilizes hES or pSC cells as the starting cells for differentiation. The embryonic stem cells used in this method can be cells that originate from the morula, embryonic inner cell mass or those obtained from embryonic gonadal ridges. The parthenogenetic stem cells used in this method originate from a pathenote. Human stem cells can be maintained in culture in a pluripotent state without substantial differentiation using methods that are known in the art. Such methods are described, for example, in U.S. Pat. Nos. 5,453,357, 5,670,372, 5,690,926 5,843,780, 6,200,806, 6,251,671 and U.S. patent application Ser. Nos. 12/082,028 and 12/629,813, the disclosures of which are incorporated herein by reference in their entireties.

The human pluripotent stem cells used herein can be maintained in culture either with or without serum. In some embodiments, serum replacement is used. In other embodiments, serum free culture techniques are used.

As used herein, “multipotent” or “multipotent cell” refers to a cell type that can give rise to a limited number of other particular cell types. As described above, endoderm cells do not differentiate into tissues produced from ectoderm or mesoderm, but rather, differentiate into the gut tube as well as organs that are derived from the gut tube. In one embodiment, the endoderm cells are derived from hESCs. Such processes can provide the basis for efficient production of human endodermal derived tissues such as pancreas, liver, lung, stomach, intestine and thyroid.

As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

In order to determine the amount of endoderm cells in a cell culture or cell population, a method of distinguishing this cell type from the other cells in the culture or in the population is desirable. Accordingly, in one embodiment, the methods further relate to cell markers whose presence, absence and/or relative expression levels are specific for definitive endoderm. As used herein, “expression” refers to the production of a material or substance as well as the level or amount of production of a material or substance. Thus, determining the expression of a specific marker refers to detecting either the relative or absolute amount of the marker that is expressed or simply detecting the presence or absence of the marker. As used herein, “marker” refers to any molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein or a small molecule.

For example, in one embodiment, the presence, absence and/or level of expression of a marker is determined by quantitative PCR (Q-PCR). Exemplary genetic markers include, but are not limited to such as FoxA2, Sox17, CXCR4, Oct4, AFP, TM, SPARC, Sox7, MIXL1, GATA4, HNF3b, GSC, FGF17, VWF, CALCR, FOXQ1, CMKOR1, CRIP1, E-cadherin, and other markers, which may be determined by quantitative Q-PCR. In another embodiment, immunohistochemistry is used to detect the proteins expressed by the above-mentioned genes. In another embodiment, Q-PCR and immunohistochemical techniques are both used to identify and determine the amount or relative proportions of such markers.

As such, it is possible to identify endoderm cells, as well as determine the proportion of endoderm cells in a cell culture or cell population. For example, in one embodiment, the definitive endoderm cells or cell populations that are produced express CXCR4, Shh, FoxA2, GSC and/or Sox17, but do not express AFP, SOX1 and/or SOX7.

As used herein, “defined-medium conditions” refer to environments for culturing cells where the concentration of components therein required for optimal growth are detailed. For example, depending on the use of the cells (e.g., therapeutic applications), removing cells from conditions that contain xenogenic proteins is important; i.e., the culture conditions are animal-free conditions or free of non-human animal proteins.

“Differentiated cell” refers to a non-embryonic, non-parthenogenetic or non-pluripotent cell that possesses a particular differentiated, i.e., non-embryonic, state. The three earliest differentiated cell types are endoderm, mesoderm, and ectoderm.

The pluripotent state of the cells used in the present invention can be confirmed by various methods. For example, the cells can be tested for the presence or absence of characteristic ES cell markers. In the case of human ES cells, examples of such markers are identified supra, and include SSEA-4, SSEA-3, TRA-1-60, TRA-1-81 and OCT 4, and are known in the art.

Also, pluripotency can be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in U.S. Pat. No. 6,642,433, incorporated by reference herein.

Yet another method of confirming pluripotency is to observe ES cell differentiation into embryoid bodies and other differentiated cell types when cultured under conditions that favor differentiation (e.g., removal of fibroblast feeder layers). This method has been utilized and it has been confirmed that the subject pluripotent cells give rise to embryoid bodies and different differentiated cell types in tissue culture.

Pluripotent cells and cell lines, included those generated using the method described herein and preferably human pluripotent cells and cell lines, have numerous therapeutic and diagnostic applications. Such pluripotent cells may be used for cell transplantation therapies or gene therapy (if genetically modified) in the treatment of numerous disease conditions.

Accordingly, in one aspect, the present invention provides a method of treating a subject utilizing cells derived from the methods described herein, wherein iPS cells are initially generated and subsequently differentiated into a specific cell type. The method includes obtaining a somatic cell from a subject; reprogramming the somatic cell into an induced pluripotent stem (iPS) cell using the methods of the invention; culturing the induced pluripotent stem (iPS) cell ex vivo to differentiate the cell into a desired cell type suitable for treating a condition; and introducing into the subject the differentiated cell, thereby treating the condition.

In a related aspect, the present invention provides a method of treating a subject utilizing cells derived from the methods described herein wherein an iPS cell is not generated initially and subsequently differentiated. The method includes contacting a cell with the nucleic acid construct, or the expression product thereof, of the invention, culturing the cell to differentiate, transdifferentiate or dedifferentiate the cell into a desired cell type suitable for treating a condition; and introducing the cell cultured cell into the subject, thereby treating the condition.

One advantage of the present invention is that it provides an essentially limitless supply of isogenic or syngenic human cells suitable for transplantation. The iPS cells are tailored specifically to the patient, avoiding immune rejection. Therefore, it will obviate the significant problem associated with current transplantation methods, such as, rejection of the transplanted tissue which may occur because of host versus graft or graft versus host rejection.

Another advantage of the present invention is that it addresses the specific problem of undifferentiated stem cells accompanying the target differentiated cells. The pSC, ES or iPS cells are capable of causing tetratomas when introduced in vivo. The presence of the nucleic acid construct, or the expression product thereof, of the invention specifically directs the pSC, ES or iPS cells to a targeted cell fate. Therefore, the invention will address the hazard associated with current stem cell culture methods and significantly enhance their safety.

The cells generated using the methods described herein may be differentiated into a number of different cell types to treat a variety of disorders by methods known in the art. For example, iPS cells may be induced to differentiate into hematopoetic stem cells, muscle cells, cardiac muscle cells, liver cells, cartilage cells, epithelial cells, urinary tract cells, neuronal cells, and the like. The differentiated cells may then be transplanted back into the patient's body to prevent or treat a condition.

The methods of the present invention can also be used in the treatment or prevention of neurological diseases. Such diseases include, for example, Alzheimer's disease, Parkinson's disease, Huntington's disease, amyotrophic lateral sclerosis (ALS), lysosomal storage diseases, multiple sclerosis, spinal cord injuries and the like.

Similarly, the cells produced in the methods of the invention can be utilized for repairing or regenerating a tissue or differentiated cell lineage in a subject. The method includes obtaining the reprogrammed cell as described herein and administering the cell to a subject (e.g., a subject having a myocardial infarction, congestive heart failure, stroke, ischemia, peripheral vascular disease, liver disease, cirrhosis, retinal disease, Parkinson's disease, Alzheimer's disease, diabetes, cancer, arthritis, various wound, immunodeficiency, aplastic anemia, anemia, and genetic disorders) and similar diseases, where an increase or replacement of a particular cell type/tissue or cellular de-differentiation is desirable. In one embodiment, the subject has damage to the tissue or organ, and the administering provides a dose of cells sufficient to increase a biological function of the tissue or organ or to increase the number of cell present in the tissue or organ. In another embodiment, the subject has a disease, disorder, or condition, and wherein the administering provides a dose of cells sufficient to ameliorate or stabilize the disease, disorder, or condition. In yet another embodiment, the subject has a deficiency of a particular cell type, such as a circulating blood cell type and wherein the administering restores such circulating blood cells.

Similarly, the cells produced in the methods of the invention can be utilized to assess the toxicity or efficacy of various agents, such as drugs, or to assess the safety of various chemical compositions, such as consumer products or as models for the study of cellular biology or various diseases.

The following examples are intended to illustrate but not limit the invention.

### Example 1

### Generation of Expression Constructs for Altering the Epigenetic Status of a Cell

This example depicts the construction of expression vectors encoding recombinant proteins have specific carboxy terminal elements. While the recombinant proteins used in this example are transcription factors, the same methods may be applied to generate any type of recombinant protein having the corboxy terminal domain described herein. In this example, the carboxy terminal domain is referred to as the HATHFUN domain as determined by the specific elements of the carboxy terminal domain using in this example. Thus recombinant transcription factor proteins are termed HATHFUN transcription factors in this and the remaining examples.

A series of protein expression constructs bearing transcription factor sequences were created for the purpose of expressing proteins that can alter the epigenetic status of cells, such as the differentiative state of cells, or to further purify a cell population to which they are applied. Possible examples include induced pluripotent stem cells, transdifferentiation from mesenchymal stem cells to endodermal cells, the forced skewing of embryoid bodies towards a targeted cell fate such as definitive endoderm or transdifferentiating fibroblasts into myocytes, chondrocytes, osteocytes and adipocytes.

The proteins were each tagged at the C-terminus with a functional domain termed HATHFUN which is composed of a first protein tag (an epitope haemagglutinin tag (HA)), a transduction domain (TAT), a second protein tag (a poly(His) domain for protein purification), a fusion domain (influenza hemagglutinin fusion peptide to enhance the transduction domain), and a nuclear localization sequence (NLS to target the protein to the nucleus). The combination of these elements added to the carboxy terminus of a protein provides an innovative protein construct for targeted alteration of a cells epigenetic status. The proteins to which the innovative carboxy terminus was added are shown in Table I, along with their accession numbers, as well as origin and modifications of the gene sequences.

TABLE I
Gene Accession Records, Source and Modifications to Transcription
Factors

| | GenBank ™ | |
|---|---|---|
| Protein | Accession Number | Origin/Modification |
| Oct4 | BC117437 | Open Biosystems |
| (POU5F1) | | |
| NANOG | BC099704 | Open Biosystems/ |
| | | Pseudogene repaired |
| Sox2 | BC013923.2 | Mr. Gene |
| Sox17 | BC140307 | Mr. Gene |
| HNF4a2 | NM_000457 | Isoform retrieved by RT-PCR |
| | | from HepG2a |
| GATA4 | BC105108 | Open Biosystems Q |
| Hhex | BU162190 | Open Biosystems |
| CEBPβ | NM_005194.2 | Origene; Q |
| CEBPδ | NM_00008.9 | Isoform retrieved by RT-PCR |
| | | from adipogenesis-induced |
| | | mesenchymal stem cells |
| MyoD1 | NM_002478.3 | Origene; Q |
| Runx2 | BC160022/NM_001024630.3 | Open Biosystems/N-terminus |
| | | 19 amino acid substitution |
| | | and Q |
| Sox9 | NM_000346.2 | Mr. Gene |

Construction of HATHFUN for use in the protein expression constructs was as follows. HATHFUN was generated from a series of four synthesized DNA primers (see Table II) that were fused together using PCR (Invitrogen, AccuPrime™ Pfx Supermix, 12344-040). They were designed to overlap with each other and generate a final sequence 187 bp long, including a stop codon and a CACC sequence at the 5′ end required for directional cloning. The finalized protein sequence was as follows.

(SEQ ID NO: 4)
YPYDVPDYAGGKKKRKVGGYGRKKRRQRRRGGHHHHHHGGGLFGAIA
GFIENGWEGMIDG

Each domain is separated by two glycines to allow them free rotation from the other domains. The fused PCR domain was cloned into the pENTR/SD/D-TOPO™ entry cloning vector following the manufacturer's instructions (Invitrogen). The cloning kit was purchased from Invitrogen (K4240-20). Upon confirmation of the presence of the insert by PCR, the construct was verified by sequences submitted to Retrogen (San Diego, Calif.).

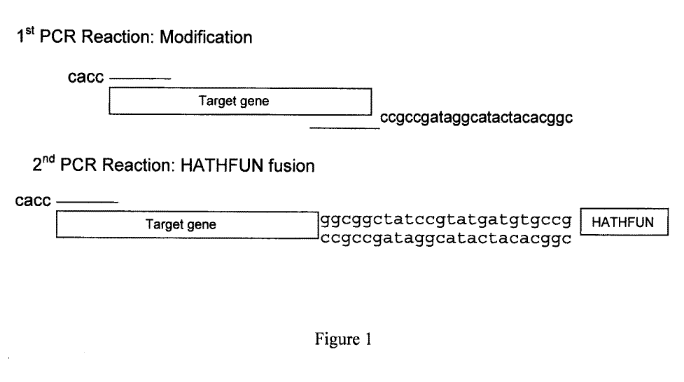

Construction of HATHFUN tagged proteins was performed using a general protocol suitable for the different proteins. Construction of the HATHFUN tagged proteins all followed a general protocol as shown in FIG. 1. Primers were designed (see Table II) in which the forward primer contained CACC at the beginning for directional cloning and the reverse primer would eliminate the stop codon present in the original endogenous sequence and provide an overlap area for fusion between the target cDNA and the HATHFUN sequence. All primers were purchased from Integrated DNA Technologies (Coralville, Iowa). The target cDNA was amplified by PCR to incorporate the modifications. Several of these cDNAs were very GC-rich or contained secondary structure and amplified only if Q solution from the Qiagen OneStep™ RT-PCR kit (210210) was present in the reaction. After verifying fragment size on agarose gel, the modified sequence was purified using QIAquick™ gel extraction kit (Qiagen, 28104). The modified sequence was then placed in a second PCR reaction with the HATHFUN sequence in which the overlap area between the two sequences would act as a priming region and fuses them together. After one cycle of amplification, the forward and reverse primers specific to the target gene and HATHFUN, respectively, were added to the reaction. The newly fused sequence, after purification, was then cloned into the pENTR/SD/D-TOPO™ vector and transformed into DH5α (Invitrogen, 18265017). Valid clones were determined using an NruI digest (New England Biolabs, R1092S) and confirmed by sequencing.

Several cDNA sequences were purchased from various companies (see Table I) which required only amplification by PCR using construct-specific primers that would allow fusion to HATHFUN with a second PCR reaction as described above. Some required repair, modification or were simply unavailable. Details of these constructs are described below.

NANOG: A pseudogene containing four point mutations compared to wild-type sequence (Accession # NM_{—}024865) was purchased from Open Biosystems. Two of the mutations result in a non-conservative amino acid change. PCR primers were designed accordingly to change the two mutations into wild-type sequence and overlapping fragments were fused into a whole. Further construction was carried out according to the general protocol outlined above.

Sox2, Sox17 and Sox9: The original mammalian sequences (accession numbers BC013923.2, BC140307 and NM_{—}000346.2, respectively) could not be expressed in the E. coli protein expression system. Optimized constructs adapted to E. coli expression were generated and purchased from Mr. Gene. Sequences are as follows.

Sox2:
(SEQ ID NO: 5)
ACCATGTATAATATGATGGAAACCGAGCTGAAACCTCCGGGTCCTCAACAAACAAG
TGGGGGTGGGGGTGGCAATAGTACTGCTGCTGCTGCTGGGGGTAACCAAAAAAACT
CTCCTGATCGTGTGAAACGCCCGATGAATGCCTTTATGGTGTGGTCACGTGGACAAC
GTCGTAAAATGGCCCAAGAGAATCCGAAAATGCACAACAGCGAGATCTCAAAACGT
CTGGGTGCTGAGTGGAAACTGCTGAGTGAAACGGAAAAACGCCCTTTCATTGACGA
AGCGAAACGCCTGCGTGCCCTGCATATGAAAGAACACCCGGACTATAAATATCGTC
CACGCCGTAAAACCAAAACCCTGATGAAAAAAGACAAATATACCCTGCCTGGTGGT
CTGCTGGCACCTGGTGGAAATTCTATGGCAAGCGGTGTCGGAGTTGGTGCTGGTCTG
GGAGCCGGTGTGAATCAGCGTATGGACTCTTATGCCCACATGAACGGTTGGAGCAA
TGGTTCCTATTCGATGATGCAAGATCAACTGGGTTATCCTCAACATCCTGGCCTGAA
TGCTCATGGAGCTGCTCAGATGCAACCGATGCACCGTTATGACGTGAGTGCACTGCA
GTATAACAGCATGACCTCTTCTCAGACCTATATGAACGGCTCACCGACTTATAGCAT
GTCGTATAGCCAACAGGGGACTCCTGGTATGGCTCTGGGTTCTATGGGTAGTGTGGT
GAAAAGCGAAGCAAGCTCTAGCCCTCCTGTGGTAACATCTTCCTCACATTCCCGTGC
CCCTTGTCAGGCTGGTGACCTGCGTGATATGATCAGCATGTATCTGCCTGGAGCAGA
AGTTCCTGAACCTGCCGCTCCTTCTCGTCTGCACATGTCCCAACATTATCAGTCTGGC
CCGGTCCCTGGAACAGCGATTAATGGTACTCTGCCTCTGTCTCACATGGGTGGATAT
CCGTATGATGTCCCGGATTATGCCGGTGGTAAAAAAAAACGTAAAGTGGGGGGTTA
TGGCCGTAAAAAACGTCGCCAACGTCGTCGTGGTGGTCATCACCATCACCATCATGG
TGGTGGCCTGTTTGGTGCTATCGCCGGCTTTATCGAAAACGGGTGGGAAGGCATGAT
TGATGGCTAA
Sox17:
(SEQ ID NO: 6)
ACCATGTCTAGCCCTGATGCCGGTTATGCCTCTGATGACCAGTCTCAAACACAGTCT
GCTCTGCCTGCCGTAATGGCCGGTCTGGGTCCTTGTCCTTGGGCCGAATCTCTGTCTC
CTATTGGCGATATGAAAGTGAAAGGCGAAGCACCAGCAAATTCTGGAGCCCCTGCC
GGAGCTGCCGGTCGTGCTAAAGGTGAATCCCGTATTCGTCGTCCGATGAACGCTTTT
ATGGTATGGGCGAAAGATGAGCGTAAACGTCTGGCACAACAGAACCCTGATCTGCA
CAATGCCGAACTGAGCAAAATGCTGGGCAAATCGTGGAAAGCACTGACTCTGGCCG
AAAAACGTCCGTTTGTGGAAGAAGCCGAGCGTCTGCGCGTACAACACATGCAAGAC
CATCCGAACTATAAATATCGTCCTCGCCGCCGTAAACAGGTTAAACGCCTGAAACGT
GTGGAGGGTGGTTTTCTGCACGGCCTGGCTGAACCTCAAGCTGCTGCCCTGGGACCT
GAAGGTGGGCGTGTAGCAATGGACGGCCTGGGACTGCAATTTCCGGAACAGGGATT
TCCAGCTGGTCCTCCTCTGCTGCCTCCTCATATGGGTGGTCACTATCGTGATTGTCAG
TCACTGGGTGCTCCACCTCTGGATGGATATCCACTGCCAACACCGGATACAAGTCCT
CTGGACGGAGTTGATCCTGATCCTGCCTTTTTTGCCGCTCCTATGCCTGGTGATTGCC
CTGCTGCTGGCACTTATTCTTATGCTCAAGTGAGCGACTATGCCGGACCTCCTGAAC
CGCCTGCCGGACCGATGCATCCTCGTCTGGGCCCTGAGCCTGCCGGGCCTTCTATTC
CTGGACTGCTGGCTCCGCCTAGTGCACTGCACGTCTATTATGGCGCTATGGGTAGCC
CTGGCGCTGGGGGTGGTCGTGGTTTTCAAATGCAACCTCAACACCAGCACCAACATC
AACATCAGCACCATCCGCCTGGTCCTGGACAACCTTCTCCTCCTCCTGAGGCACTGC
CTTGTCGTGATGGGACGGATCCTTCTCAACCTGCTGAACTGCTGGGTGAAGTCGATC
GTACCGAATTCGAACAGTATCTGCACTTTGTGTGTAAACCGGAGATGGGTCTGCCAT
ATCAAGGTCATGATAGCGGCGTTAATCTGCCGGATTCTCATGGCGCTATTAGCAGCG
TTGTTTCCGATGCCAGTAGTGCCGTGTATTATTGTAACTATCCGGATGTGGGTGGCTA
TCCTTATGATGTGCCTGATTATGCCGGTGGTAAAAAAAAACGTAAAGTGGGCGGCTA
TGGTCGTAAAAAAC GTCGTCAGCGTCGTCGTGGGGGACACCATCATCATCATCATGG
TGGGGGACTGTTTGGCGCTATTGCCGGCTTTATCGAAAATGGCTGGGAAGGCATGAT
TGATGGCTAA
Sox9:
(SEQ ID NO: 7)
ACCATGAACCTGCTGGACCCTTTTATGAAAATGACCGACGAACAGGAGAAAGGTCT
GTCTGGAGCACCTTCACCAACCATGTCCGAAGATTCTGCCGGTAGTCCTTGCCCTAG
TGGTAGTGGTAGTGACACGGAAAACACACGTCCTCAAGAGAACACGTTCCCGAAAG
GCGAACCTGATCTGAAAAAAGAGAGCGAGGAGGACAAATTTCCGGTTTGTATCCGT
GAAGCAGTGAGCCAAGTGCTGAAAGGATATGACTGGACGCTGGTTCCTATGCCAGT
TCGTGTGAATGGCAGCTCCAAAAACAAACCTCACGTGAAACGTCCAATGAATGCCTT
CATGGTGTGGGCACAAGCAGCACGTCGTAAACTGGCTGACCAGTATCCACATCTGC
ATAACGCTGAACTGAGCAAAACACTGGGGAAACTGTGGCGTCTGCTGAATGAAAGC
GAGAAACGCCCTTTTGTAGAAGAAGCCGAACGCCTGCGCGTACAACACAAAAAAGA
CCACCCGGACTATAAATATCAGCCTCGCCGCCGTAAAAGTGTGAAAAACGGCCAGG
CCGAAGCAGAGGAAGCAACAGAACAGACACACATTAGCCCGAATGCCATCTTTAAA
GCCCTGCAGGCAGACTCACCTCATAGCAGTAGTGGAATGAGCGAAGTCCATAGCCC
TGGAGAACATTCTGGACAGTCTCAAGGCCCTCCAACACCTCCGACAACCCCAAAAA
CTGACGTTCAACCGGGTAAAGCTGACCTGAAACGTGAAGGACGTCCACTGCCAGAA
GGTGGTCGTCAACCTCCAATCGATTTTCGTGACGTGGACATTGGCGAGCTGTCTAGT
GATGTGATCAGCAATATCGAAACCITCGATGTTAACGAGTTCGACCAATATCTGCCG
CCAAATGGTCATCCTGGTGTTCCGGCTACACATGGACAAGTGACCTATACGGGCTCA
TATGGTATTAGCAGTACCGCCGCTACACCTGCCTCAGCTGGGCATGTTTGGATGTCG
AAACAGCAAGCACCGCCTCCGCCTCCACAACAACCGCCTCAAGCACCTCCGGCCCC
TCAGGCACCGCCTCAACCTCAAGCAGCCCCTCCACAACAACCTGCCGCTCCGCCTCA
ACAGCCTCAAGCCCATACACTGACAACCCTGTCTAGTGAACCTGGACAGTCTCAGCG
TACCCACATTAAAACCGAGCAGCTGTCACCGTCACATTATAGCGAACAGCAACAGC
ATAGCCCTCAGCAAATTGCCTATTCCCCGTTCAATCTGCCACACTATTCACCATCGTA
TCCGCCGATTACTCGTAGTCAGTATGACTATACCGATCACCAGAACAGTTCCTCGTA
TTATAGCCATGCCGCCGGTCAGGGTACAGGACTGTATAGCACCTTCACATATATGAA
TCCGGCACAACGTCCGATGTATACCCCGATTGCCGATACTAGTGGAGTTCCGAGCAT
TCCTCAGACCCATAGCCCTCAACATTGGGAACAGCCGGTCTATACCCAACTGACACG
CCCTGGGGGTTATCCGTATGATGTCCCAGATTATGCCGGGGGTAAAAAAAAACGTA
AAGTGGGCGGGTATGGTCGTAAAAAACGCCGTCAACGCCGCCGTGGTGGCCATCAT
CACCACCATCATGGAGGAGGCCTGTTTGGCGCTATTGCCGGCTTTATTGAGAATGGG
TGGGAAGGCATGATTGATGGCTAA

HNF4α2: Many variations of the HNF4 gene exist, driven by two distinct promoters. Very few are commercially available. This particular isoform (HNF4α2) was reported to be expressed in hepatocytes. One of the immortal hepatic lines, HepG2a, was also reported to express this protein. The HepG2a cell line was purchased from ATCC(CRL-10741) and grown according to manufacturer's instructions. Primers were designed to retrieve by RT-PCR this particular isoform. RNA was extracted using the RNeasy Plus Mini™ kit (Qiagen, 74134) then subjected to RT-PCR according to manufacturer's directions (kit described above). After verifying fragment size by agarose gel, further construction was carried out according to the general protocol outlined above.

Runx2: Of three reported isoforms, only one was commercially available. The available isoform lacked 19 unique amino acids at the N-terminus which may confer function specific to osteocyte differentiation, although this requires experimental verification in our system. This longer isoform is also referred to as OSF2/CBFA1a. The shorter isoform was purchased. A series of PCR primers were designed to substitute the original five amino acids of the N-terminal domain for the new isoform-specific ones and allow fusion to the original sequence. After creating the elongated sequence using a series of PCR reactions, further construction was carried out according to the general protocol outlined above.

CEBPδ: This gene was not commercially available. The protein is expressed during the differentiation of mesenchymal stem cells into adipocytes. Primers were designed to retrieve by RT-PCR this gene. Mesenchymal stem cells isolated in our laboratory were induced to undergo adipogenesis. RNA was extracted using the RNeasy Plus Mini™ kit (Qiagen, 74134) then subjected to RT-PCR according to manufacturer's directions (kit described above). After verifying fragment size by agarose gel, further construction was carried out according to the general protocol outlined above.

TABLE II
Primers Generated for Construct Modification and
Tagging

| | | SEQ |
|---|---|---|
| Gene | Primers(forward/reverse) | ID NO |
| HATHFUN | agaggaaggtgggcggctatggccgcaaaaaa | 8 |
| Primer1 | cgccgccagcgccgccgcggcgg | |
| HATHFUN | caatcgcgccaaacaggccgccgccatgatga | 9 |
| Primer2R | tgatgatgatggccgccgcggcg | |
| HATHFUN | cacctatccgtatgatgtgccggattatgcgg | 10 |
| Primer 3 | gcggcaagaagaagaggaaggtg | |
| HATHFUN | ttagccatcaatcatgccttcccagccgtttt | 11 |
| Primer 4R | caataaagcccgcaatcgcgcca | |
| HATHFUN | ggcggctatccgtatgatgtgccggat | 12 |
| Primer | | |
| 5Ext | | |
| Oct4 | caccatggcgggacacctggcttc/ | 13, 14 |
| (POU5F1) | cggcacatcatacggatagccgccgtttgaat | |
| | gcatgggagagc | |
| NANOG | caccatgagtgtggatccagcttg/ | 15, 16 |
| | cggcacatcatacggatagccgcccacgtctt | |
| | caggttgcatgt | |
| NanogT:G | cttctgcagagaagagtgtcgcaaaa/ttttg | 17, 18 |
| Pt | cgacactcttctctgcagaag | |
| mut 394 | | |
| NanogC:G | gtcctgcatgcagttccagccaaatt/ | 19, 20 |
| Pt | aatttggctggaactgcatgcaggac | |
| mut 907 | | |
| Sox2 | caccatgtataatatgatggaaaccg/ | 21, 22 |
| | ttagccatcaatcatgcctt | |
| Sox17 | caccatgtctagccctgatgc/ | 23, 24 |
| | ttagccatcaatcatgcctt | |
| HNF4a2 | caccatgcgactctccaaaaccct/cggca | 25, 26 |
| | catcatacggatagccgccgataacttcct | |
| | gcttggtga | |
| GATA4 | caccatgtatcagagcttggccat/ | 27, 28 |
| | cggcacatcatacggatagccgcccgcagtga | |
| | ttatgtccccgt | |
| Hhex | caccatgcagtacccgcaccccgg/ | 29, 30 |
| | cggcacatcatacggatagccgcctccagcat | |
| | taaaatagcttt | |
| CEBPβ | caccatgcaacgcctggtggcctg/ | 31, 32 |
| | cggcacatcatacggatagccgccgcagtggc | |
| | cggaggaggcga | |
| MyoD1 | caccatggagctactgtcgccacc/ | 33, 34 |
| | cggcacatcatacggatagccgccgagcacct | |
| | ggtatatcgggt | |
| Runx2 | caccatggcatcaaacagcctctt/ | 35, 36 |
| | cggcacatcatacggatagccgccatatggtc | |
| | gccaaacagatt | |
| Runx2 | acaccatgtcagcaaaacttcttttgggatcc | 37 |
| 19aa F1 | gagcaccagccggcg | |
| Runx2 | atggcatcaaacagcctcttcagcacagtgac | 38 |
| 19aa F2 | accatgtcagcaaaactt | |
| Sox9 | caccatgaacctggacccttt/ | 39, 40 |
| | ttagccatcaatcatgcctt | |
| CEBPδ | caccatgagcgccgcgctcttcag/ | 41, 42 |
| | cggcacatcatacggatagccgccccggcagt | |
| | ctgctgtcccgg | |

Protein expression and purification was performed as follows. Fused expression sequences were recombined into the pBAD-DEST protein expression vector (Invitrogen, 12283-016) using the GATEWAY technology and transformed into TOP10 (included in the kit). Valid clones were determined using a NruI digest. Protein expression was confirmed by inducing freshly seeded bacterial cultures with 0.2% L-arabinose (Sigma, A91906) and running on NuPAGE Novex 10% Bis-Tris acrylamide gel (Invitrogen, NP0302BOX).

Once protein expression was confirmed, large scale culture was initiated. As a general protocol, cultures were centrifuged 1500×g, washed once with PBS and frozen at −80° C. until processed. Pellets were thawed in guanidine HCl buffer (6 M GnHCl, 500 mM NaCl, 20 mM Tris-HCl pH 8.0, 20 mM imidazole) and lysed three times using nitrogen decompression (1700 psi, Parr Instrument Company). Lysate was run over Gravitrap™ affinity columns purchased from GE Lifesciences (28-4013-51), washed twice with urea buffer (8 M urea, 250 mM NaCl, 20 mM Tris-HCl, 20 mM imidazole) then eluted in fractions using urea buffer with increasing amounts of imidazole (50 mM, 100 mM, 250 mM, 500 mM). Fractions were individually collected and run on acrylamide gel to locate the target protein.

Positive fractions were pooled then further concentrated using ultrafiltration centrifugation (Millipore, UFC901008).

Concentrated proteins were run on PD10 desalting columns (GE Lifesciences, 17-0851-01) according to manufacturer's instructions to exchange the urea buffer to phosphate buffered saline, Gly-gly buffer (25 mM Tris-HCl pH 7.4, 10% glycerol, 100 mM glycine), 500 mM Arginine-HCl or other buffer which keeps the target protein soluble. Final protein concentration was determined using BCA protein assay (Thermo Scientific Fisher, 23227).

### Example 2

### Differentiation of hESCs to Hepatocytes

Purification of hESC culture by applying transducible HATHFUN-tagged Oct4, Nanog and Sox2 proteins to hESCs. Embryonic stem cells are a heterogenous culture of cells with the purest pluripotent stem cells at the center. It has been previously demonstrated that the application of transducible Oct4 or Sox2 to murine ESCs grown on gelatin for five days was sufficient to enhance the purity of the mESCs from 14% to 68% and 56%, respectfully. HATHFUN-tagged Oct4, NANOG and Sox2, in the presence of aprotinin (protease inhibitor) will be applied to hESCs grown on Matrigel™ or other coated surface then assayed by flow cytometry for the presence of markers such as TRA-1-60 and SSEA-4 to determine the effects of the proteins. Proteins will be applied to the cells by two different methods to assess which is most effective (FIG. 2). The first method applies proteins directly to the cell culture for a span of five days then monitors culture purity for three passages to determine the half-life of the effects. The second method applies proteins to cells only during passaging of the culture for three passages, taking advantage of the “stickiness” that is characteristic of tranduction-domain bearing proteins. This method would serve to reduce the amount of protein required. Also, since these proteins can convert fibroblasts into iPS cells, it is possible that a mitomycin C-treated fibroblast feeder layer could be converted, despite the genetic damage inflicted by the mitomycin C. The protein treatment during passaging would therefore demonstrate an alternative methodology if an hESC line requires a fibroblast feeder layer. Later refinements to this process would possibly include the use of Accutase to enhance the size uniformity of the hESC colonies, the creation of spheroids using microwell rotation or Aggrewells™ (Stem Cell, 27845) to further enhance the purity of the hESC culture.

TABLE 2
Experimental Design to Measure HATHFUN-tagged Protein Effects

| | | |
|---|---|---|
| Control | Treat vs fadeout | Treat at each passage three |
| | | times |
| No treatment | Treat in well with fluid | Harvest each passage for |
| | change every 24 hours | three passages |
| | for 5 days | |
| | Stop treatment and harvest | |
| | at each passage for 3 passages | |

Creation of definitive endoderm may be performed as follows. The creation of embryoid bodies from hESCs generates three primordial tissue types: endodermal, mesoderm and ectodermal. The organization of early embryoid bodies (EBs) usually has an outer layer of endoderm with an inner core of ESCs and ectoderm. To date very few references discuss control for size of aggregates or EBs as a means of directing differentiation. According to one reference, small aggregates are vitally important to direct cells toward an endodermal pathway. Furthermore, others clearly demonstrates that Sox17 is a vital signal for the establishment of definitive endoderm.

The use of HATHFUN-tagged Sox17 in combination with disaggregation and uniformly small embryoid body sizes should markedly alter the cell fates to a majority of endodermal cells and further specify them as definitive (as opposed to extraembryonic) endoderm.

Two possible protocols may be followed. The first is the establishment of a monolayer culture of pure definitive endodermal stem cells which may be maintained or frozen for later use. The second relies on the use of spheroid culture to enable the intrinsic signaling mechanisms present for differentiation when cells are in a three-dimensional format.

Protocol 1 is performed as follows. Differentiation of purified hESCs to definitive endoderm will be begun by creating a single cell suspension using Accutase to ensure all cells receive equal amounts of signal. Cells would be treated only with HATHFUN-Sox17 to enable the largest retention of multipotent cells. Cells would then be plated and maintained in conditioned embryonic stem cell media. Further treatments may or may not be necessary depending on the purity of the input hESCs and the ability of the conditioned ESC media to maintain multipotency. Assays to characterize definitive endoderm are described below. If pure definitive endodermal cells can be maintained, this will serve as a cell bank for further differentiation.

Protocol 2 is performed as follows. The following protocol is an amalgamation derived from several laboratories and additionally incorporates the use of HATHFUN-Sox17 and Aggrewells™. Similar to Protocol 1, differentiation of purified hESCs to definitive endoderm will be begun by creating a single cell suspension using Accutase to ensure all cells receive equal amounts of signal. Alternatively, cells derived from Protocol 1 will be used. HATHFUN-tagged Sox17 protein and Activin A will be applied to the cell suspension, rinsed, and then plated into Aggrewells™ to form endodermal EBs of −250 cells/EB. Medium is Knockout™ DMEM, bFGF, Activin A, Wnt3a, and Knockout™ Serum Replacement (Invitrogen).

After incubation for 2 days in the Aggrewell™, a second treatment to sustain endodermal differentiation toward the hepatic pathway is performed. The endodermal EBs are digested with Accutase, treated again with HATHFUN-tagged Sox 17 protein and Activin A and reaggregated in Aggrewells™. EBs are incubated for 2 more days in the same medium.

Purity of endodermal cells can be assayed by using cell surface markers such as CXCR4 and c-kit by flow cytometry, or internal markers such as Shh, Foxa2, goosecoid and lack of AFP (α-fetoprotein), Sox7 (primitive endoderm marker) and Sox1 (ectoderm) by immunocytochemistry.

The number of dissociation and reaggregations might be reduced depending on how loose the EBs and spheroids are. The accessibility of the inner core of cells to signals will determine the need for dissociation. After the initial experiments, variations in spheroid size and number of dissociations/aggregations versus success rate can be attempted.

Hepatoblast/biphasic differentiation may be performed as follows. This phase will initiate the change from endoderm to neonatal hepatoblasts. This is the first known attempt to aid in the generation and maturation of hepatocytes by utilizing spheroid culture. Many references in the literature indicate that culturing in vivo derived hepatocytes in a spheroid format results in the most long-lived cultures with the required drug metabolizing enzymes compared to 2D sandwich culture. It is hypothesized that the generation of a 3D format in combination with our HATHFUN-tagged transcription factors will aid the creation and function of these cells in several respects.

Differentiation to hepatoblast/biphasic stage will be initiated by creating a single cell suspension using Accutase to ensure all cells receive equal amounts of signal. Suspension will be treated with HATHFUN-tagged HNF4α2, GATA4, Hhex plus Activin A in hepatoblast medium. Suspension will be aggregated via Aggrewell™ and incubated for 1 day.

If any sizable population of non-target cells exists, cells may be sorted before treatment for CXCR4+ cells using MACS column.

Maturation of hepatocytes may be performed as follows. This stage of culture is meant to allow further maturation of the hepatocytes and allow their handling in bulk culture. Spheroids are removed from Aggrewells™, and then coated to prevent fusion and/or aid in maturation. The coatings may be alginate, extracellular matrix (ECM) such as a mix of collagen VIII, fibronectin and dermatan sulphate proteoglycans (DSPG) or ECM-doped alginate. Coated spheroids are incubated in hepatocyte differentiation/maturation medium. Other possible maturation factors known in the art may be included, such as Oncostatin M and/or bile salt.

Functional assays for hepatic function will include albumin and fibronectin secretion, presence of glycogen storage (Schiff acid stain), Cyp3A4 and Cyp7A1 (adult only P450) versus adult human hepatocytes.

### Example 3

### Differentiation of Mesoderm to Endoderm

The purpose of this experiment is to demonstrate that mesoderm can be transdifferentiated into definitive endoderm using the HATHFUN-tagged Sox17 protein. Adipose-derived mesodermal stem cells are treated with the HATHFUN-tagged Sox17 protein for a minimum of 2-3 days and observed daily for morphological changes. The culture media is a standard ESC medium such as Knockout™ DMEM/Knockout™ Serum Replacement. ESC-conditioned medium may also be added to the culture to aid in the transdifferentiation. In addition to morphological changes, cells would be further assayed using the markers described above for the endodermal lineage.

### Example 4

### Differentiation of Mesoderm or Fibroblasts to Adiptocytes, Chondryocytes, Osteocytes and Myocytes

The ability to differentiate mesoderm or fibroblasts into adipocytes, chondrocytes, osteocytes and myocytes has been repeatedly demonstrated in the literature and the protocols well documented (see Chen et al., Journal of Cell Science (2007) 120:2875-83; and Bartsch et al., Stem Cells and Development (2005) 14:337-348).

However, the efficiency of these protocols are quite limited with only a small percentage of the target cells actually retaining multipotency and a minority able to differentiate into only one or two tissue types (see Chen et al. and Bartsch et al.). A search of the literature was made for the unique transcription factors that directly control differentiation into the target cells. While several have been reported at different stages of differentiation or show multiple functions, those factors selected included CEBPβ/CEBPδ (adipocytes), Sox9 (chondrocytes), Runx2 (osteocytes) and MyoD1 (myocytes) and were tagged with HATHFUN.

Differentiation kits may be purchased commercially or generated. All four target cell types require approximately three weeks for full differentiation. Adipose-derived mesenchymal cells or foreskin fibroblasts will be treated with differentiation kits and the HATHFUN-tagged proteins applied during the treatment. Subsequent assays for differentiation will be Oil Red O stain (adipocytes), Alcain Blue (chondrocytes), Alizarin Red (osteocytes) and multinucleated fused myotubes (myocytes).

Although the invention has been described with reference to the above examples, it will be understood that modifications and variations are encompassed within the spirit and scope of the invention. Accordingly, the invention is limited only by the following claims.

What is claimed is:

1. A method of reprogramming, differentiating, transdifferentiating or dedifferentiating a target or recipient cell into a cell of a different cell type or into a pluripotent cell or less differentiated cell by introducing one or more nucleic acid constructs into the cell, or the expression product thereof, and culture under culture conditions that convert the cell into a pluripotent cell or into a cell of a cell lineage corresponding to endoderm, mesoderm or ectoderm.

2. The method of claim 1, wherein the construct encodes a cassette comprising in operable linkage:
i) at least one protein tag;
ii) a protein transduction domain;
iii) a fusion domain;
iv) a nuclear localization signal; and
v) at least one transcription factor.

3. The method of claim 2, wherein the transcription factor is a nuclear reprogramming factor.

4. The method of claim 3, wherein the transcription factor is encoded by a gene selected from the group consisting of a SOX family gene, a KLF family gene, a MYC family gene, SALL4, OCT4, NANOG, LIN28, or a combination thereof.

5. The method of claim 4, wherein the transcription factor is Oct4, Sox2, Klf4, Nanog, or c-Myc.

6. The method of claim 2, wherein the transcription factor is encoded by a gene selected from the group consisting of OCT4, NANOG, SOX2, SOX17, HNF4, GATA4, HHEX, CEBPβ, CEBPδ, PRDM16, MYOD1, NKX2.5, MEF2c, MYOCARDIN, RUNX2, PDX, NGN, SALL4 or SOX9.

7. The method of claim 6, wherein the transcription factor is Oct4, Nanog, Sox2, Sox9, Sox17, HNF4α2, HNF4α4, HNF4α7, HNF4α8, HNF4γ, GATA4, Hhex, CEBPβ, CEBPδ, PRDM16, MyoD1, NKX2.5, Mef2c, Myocardin, Runx2-I, Pdx1, Ngn3, Sall4 or Runx2-II.

8. The method of claim 1, wherein the cell is undifferentiated, partially differentiated, or fully differentiated before the nucleic acid construct, or the product thereof is introduced into the cell.

10. The method of claim 1, wherein the cell is an embryonic stem (ES) cell, a pluripotent stem (PS) cell, an induced pluripotent stem (iPS) cell, a parthenogenetic stem cell, a mesenchymal stem cell, a mesodermal stem cell, an endodermal stem cell, an ectodermal stem cell, a multipotent stem cell, a bipotent stem cell, a somatic stem cell, or a somatic cell.

11. The method of claim 10, wherein the endodermal stem cell expresses one or more markers selected from the group consisting of FoxA2, Sox17, CXCR4, brachyury, and CER1.

12. The method of claim 11, wherein the endodermal stem cell is a hepatocyte, cholangiocyte, pancreatic exocrine or endocrine beta-cell, or the mesodermal stem cell is an adipocyte, chondrocyte, osteocyte, or myocyte.

13. The method of claim 1, wherein the cell is cultured in the presence of one or more maturation factors.

14. The method of claim 2, wherein the nucleic acid construct comprises at least two protein tags.

15. The method of claim 2, wherein the nucleic acid construct comprises three or more protein tags.

16. The method of claim 14, wherein the at least two protein tags comprise an affinity tag and an epitope tag.

17. The method of claim 14, wherein the at least two protein tags comprise a poly(His) tag and a haemagglutinin (HA) epitope tag.

18. The method of claim 1, wherein the at least one protein tag is selected from the group consisting of poly(His), haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO) and ubiquitin (Ub).

19. The method of claim 2, wherein the fusion domain comprises influenza hemagglutinin fusion peptide or fragment thereof.

20. The method of claim 2, wherein the protein transduction domain comprises a TAT protein, VP22 protein, Drosophila Antennapedia (Antp) homeotic transcription factor, or fragments thereof.

21. The method of claim 1, wherein each of (i) to (iv) are separated by 1 to 10 amino acids.

22. The method of claim 21, wherein each of (i) to (iv) are spaced by 2 amino acids.

23. The method of claim 22, wherein the amino acids are glycine.

24. The method of claim 2, wherein the nucleic acid construct encodes at least one additional cassette comprising in operable linkage:
i) at least one protein tag;
ii) a protein transduction domain;
iii) a fusion domain;
iv) a nuclear localization signal; and
v) at least one transcription factor.

25. The method of claim 1, wherein at least one additional nucleic acid construct, or expression product thereof, is introduced into the cell, wherein the at least one additional construct encodes in operable linkage:
i) at least one protein tag;
ii) a protein transduction domain;
iii) a fusion domain;
iv) a nuclear localization signal; and
v) at least one transcription factor.

26. A nucleic acid construct encoding a cassette comprising in operable linkage:
i) at least one protein tag;
ii) a protein transduction domain;
iii) a fusion domain;
iv) a nuclear localization signal; and
v) a transcription factor.

27. The nucleic acid construct of claim 26, wherein the transcription factor is a nuclear reprogramming factor.

28. The method of claim 27, wherein the transcription factor is encoded by a gene selected from the group consisting of a SOX family gene, a KLF family gene, a MYC family gene, SALL4, OCT4, NANOG, LIN28, or a combination thereof.

29. The method of claim 28, wherein the transcription factor is Oct4, Sox2, Klf4, Nanog, or c-Myc.

30. The method of claim 26, wherein the transcription factor is encoded by a gene selected from the group consisting of OCT4, NANOG, SOX2, SOX17, HNF4, GATA4, HHEX, CEBPβCEBPδ, PRDM16, MYOD1, NKX2.5, MEF2c, MYOCARDIN, RUNX2, SALL4 or SOX9.

31. The method of claim 30, wherein the transcription factor is Oct4, NANOG, Sox2, Sox9, Sox17, HNF4α2, HNF4α4, HNF4γ, GATA4, Hhex, CEBPβ, CEBPδ, PRDM16, MyoD1, NKX2.5, Mef2c, Myocardin, Runx2-I, Sall4 or Runx2-II.

31. The nucleic acid construct of claim 26, wherein the nucleic acid construct comprises at least two protein tags.

32. The nucleic acid construct of claim 31, wherein the at least two protein tags comprise an affinity tag and an epitope tag.

33. The nucleic acid construct of claim 31, wherein the at least two protein tags comprise a poly(His) tag and a haemagglutinin (HA) epitope tag.

34. The nucleic acid construct of claim 26, wherein the at least one protein tag is selected from the group consisting of poly(His), haemagglutinin (HA) epitope, myc epitope, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin (TRX), poly(NANP), V5, S-protein, streptavidin, SBP, poly(Arg), DsbA, c-myc-tag, HAT, cellulose binding domain, softag 1, softag3, small ubiquitin-like modifier (SUMO) and ubiquitin (Ub).

35. The nucleic acid construct of claim 26, wherein the fusion domain comprises influenza hemagglutinin fusion peptide or fragment thereof.

36. The nucleic acid construct of claim 26, wherein the protein transduction domain comprises a TAT protein, VP22 protein, Drosophila Antennapedia (Antp) homeotic transcription factor, or fragments thereof.

37. The nucleic acid construct of claim 26, wherein each of (i) to (iv) are separated by 1 to 10 amino acids.

38. The nucleic acid construct of claim 37, wherein each of (i) to (iv) are spaced by 2 amino acids.

39. The nucleic acid construct of claim 38, wherein the amino acids are glycine.

40. The nucleic acid construct of claim 26, wherein the nucleic acid construct encodes a second cassette comprising in operable linkage:
i) at least one protein tag;
ii) a protein transduction domain;
iii) a fusion domain; and
iv) a nuclear localization signal; and
v) at least one transcription factor.

41. An expression vector comprising the construct of claim 26.

42. An isolated protein encoded by the nucleic acid construct of claim 26.

43. A method of enhancing retention of pluripotentcy of a parthenogenic stem cells using an isolated protein encoded by the nucleic acid construct of claim 26.

44. A method of enhancing retention of pluripotentcy of an embryonic stem cells using an isolated protein encoded by the nucleic acid construct of claim 26.

45. A method of differentiating unregulated ES, iPS or parthenogenic stem cells that are otherwise unresponsive to differentiation signals using an isolated protein encoded by the nucleic acid construct of claim 26.

46. A method of enhancing differentiation, transdifferentiation or dedifferentiation of cells using an isolated protein encoded by the nucleic acid construct of claim 26.

47. A method of treating a subject comprising:
a) obtaining a somatic cell from a subject;
b) reprogramming the somatic cell into an induced pluripotent stem (iPS) cell using the method of claim 1;
c) culturing the induced pluripotent stem (iPS) cell ex vivo to differentiate the cell into a desired cell type suitable for treating a condition; and
d) introducing into the subject the differentiated cell, thereby treating the condition.

48. The method of claim 47, wherein differentiation of the iPS cell in (c) is performed using the method of claim 1.

49. A method of treating a subject comprising:
a) contacting a cell with the construct of claim 26, or expression product thereof,
b) culturing the cell to differentiate, transdifferentiate or dedifferentiate the cell into a desired cell type suitable for treating a condition; and
c) introducing into the subject the cell of (b), thereby treating the condition.

50. The method of claim 49, wherein differentiation, transdifferentiation or dedifferentiation of the cell in (b) is performed using the method of claim 1.

51. A method of performing a cell-based assay comprising:
a) contacting a cell with the construct of claim 26, or expression product thereof,
b) culturing the cell to differentiate, transdifferentiate or dedifferentiate the cell into a desired cell type suitable for treating a condition; and
c) exposing the cell of (b) to an agent; and
d) detecting the effect of the agent on the cell.

52. The method of claim 51, wherein the agent is a drug or chemical composition.

53. The method of claim 51, further comprising utilizing the cells of (b) for in vitro cellular assays and modeling systems.


SEQUENCE LISTING (44 sequences)

SEQ ID NO: 1 - protein, 11 aa, Artificial Sequence
  Synthetic construct
YGRKKRRQRRR

SEQ ID NO: 2 - protein, 20 aa, Artificial Sequence
  Synthetic construct
GLFGAIAGFIENGWEGMIDG

SEQ ID NO: 3 - protein, 6 aa, Artificial Sequence
  Synthetic construct
KKKRKV

SEQ ID NO: 4 - protein, 60 aa, Artificial Sequence
  Synthetic construct
YPYDVPDYAGGKKKRKVGGYGRKKRRQRRRGGHHHHHHGGGLFGAIAGFIENGWEGMIDG

SEQ ID NO: 5 - DNA, 1143 bp, Artificial Sequence
  Synthetic construct
accatgtataatatgatggaaaccgagctgaaacctccgggtcctcaacaaacaagtgggggtgggggtggcaatagtactgctgctgctgctgggggtaaccaaaaaaactctcctgatcgtgtgaaacgcccgatgaatgcctttatggtgtggtcacgtggacaacgtcgtaaaatggcccaagagaatccgaaaatgcacaacagcgagatctcaaaacgtctgggtgctgagtggaaactgctgagtgaaacggaaaaacgccctttcattgacgaagcgaaacgcctgcgtgccctgcatatgaaagaacacccggactataaatatcgtccacgccgtaaaaccaaaaccctgatgaaaaaagacaaatataccctgcctggtggtctgctggcacctggtggaaattctatggcaagcggtgtcggagttggtgctggtctgggagccggtgtgaatcagcgtatggactcttatgcccacatgaacggttggagcaatggttcctattcgatgatgcaagatcaactgggttatcctcaacatcctggcctgaatgctcatggagctgctcagatgcaaccgatgcaccgttatgacgtgagtgcactgcagtataacagcatgacctcttctcagacctatatgaacggctcaccgacttatagcatgtcgtatagccaacaggggactcctggtatggctctgggttctatgggtagtgtggtgaaaagcgaagcaagctctagccctcctgtggtaacatcttcctcacattcccgtgccccttgtcaggctggtgacctgcgtgatatgatcagcatgtatctgcctggagcagaagttcctgaacctgccgctccttctcgtctgcacatgtcccaacattatcagtctggcccggtccctggaacagcgattaatggtactctgcctctgtctcacatgggtggatatccgtatgatgtcccggattatgccggtggtaaaaaaaaacgtaaagtggggggttatggccgtaaaaaacgtcgccaacgtcgtcgtggtggtcatcaccatcaccatcatggtggtggcctgtttggtgctatcgccggctttatcgaaaacgggtgggaaggcatgattgatggctaa

SEQ ID NO: 6 - DNA, 1434 bp, Artificial Sequence
  Synthetic construct
accatgtctagccctgatgccggttatgcctctgatgaccagtctcaaacacagtctgctctgcctgccgtaatggccggtctgggtccttgtccttgggccgaatctctgtctcctattggcgatatgaaagtgaaaggcgaagcaccagcaaattctggagcccctgccggagctgccggtcgtgctaaaggtgaatcccgtattcgtcgtccgatgaacgcttttatggtatgggcgaaagatgagcgtaaacgtctggcacaacagaaccctgatctgcacaatgccgaactgagcaaaatgctgggcaaatcgtggaaagcactgactctggccgaaaaacgtccgtttgtggaagaagccgagcgtctgcgcgtacaacacatgcaagaccatccgaactataaatatcgtcctcgccgccgtaaacaggttaaacgcctgaaacgtgtggagggtggttttctgcacggcctggctgaacctcaagctgctgccctgggacctgaaggtgggcgtgtagcaatggacggcctgggactgcaatttccggaacagggatttccagctggtcctcctctgctgcctcctcatatgggtggtcactatcgtgattgtcagtcactgggtgctccacctctggatggatatccactgccaacaccggatacaagtcctctggacggagttgatcctgatcctgccttttttgccgctcctatgcctggtgattgccctgctgctggcacttattcttatgctcaagtgagcgactatgccggacctcctgaaccgcctgccggaccgatgcatcctcgtctgggccctgagcctgccgggccttctattcctggactgctggctccgcctagtgcactgcacgtctattatggcgctatgggtagccctggcgctgggggtggtcgtggttttcaaatgcaacctcaacaccagcaccaacatcaacatcagcaccatccgcctggtcctggacaaccttctcctcctcctgaggcactgccttgtcgtgatgggacggatccttctcaacctgctgaactgctgggtgaagtcgatcgtaccgaattcgaacagtatctgcactttgtgtgtaaaccggagatgggtctgccatatcaaggtcatgatagcggcgttaatctgccggattctcatggcgctattagcagcgttgtttccgatgccagtagtgccgtgtattattgtaactatccggatgtgggtggctatccttatgatgtgcctgattatgccggtggtaaaaaaaaacgtaaagtgggcggctatggtcgtaaaaaacgtcgtcagcgtcgtcgtgggggacaccatcatcatcatcatggtgggggactgtttggcgctattgccggctttatcgaaaatggctgggaaggcatgattgatggctaa

SEQ ID NO: 7 - DNA, 1719 bp, Artificial Sequence
  Synthetic construct
accatgaacctgctggacccttttatgaaaatgaccgacgaacaggagaaaggtctgtctggagcaccttcaccaaccatgtccgaagattctgccggtagtccttgccctagtggtagtggtagtgacacggaaaacacacgtcctcaagagaacacgttcccgaaaggcgaacctgatctgaaaaaagagagcgaggaggacaaatttccggtttgtatccgtgaagcagtgagccaagtgctgaaaggatatgactggacgctggttcctatgccagttcgtgtgaatggcagctccaaaaacaaacctcacgtgaaacgtccaatgaatgccttcatggtgtgggcacaagcagcacgtcgtaaactggctgaccagtatccacatctgcataacgctgaactgagcaaaacactggggaaactgtggcgtctgctgaatgaaagcgagaaacgcccttttgtagaagaagccgaacgcctgcgcgtacaacacaaaaaagaccacccggactataaatatcagcctcgccgccgtaaaagtgtgaaaaacggccaggccgaagcagaggaagcaacagaacagacacacattagcccgaatgccatctttaaagccctgcaggcagactcacctcatagcagtagtggaatgagcgaagtccatagccctggagaacattctggacagtctcaaggccctccaacacctccgacaaccccaaaaactgacgttcaaccgggtaaagctgacctgaaacgtgaaggacgtccactgccagaaggtggtcgtcaacctccaatcgattttcgtgacgtggacattggcgagctgtctagtgatgtgatcagcaatatcgaaaccttcgatgttaacgagttcgaccaatatctgccgccaaatggtcatcctggtgttccggctacacatggacaagtgacctatacgggctcatatggtattagcagtaccgccgctacacctgcctcagctgggcatgtttggatgtcgaaacagcaagcaccgcctccgcctccacaacaaccgcctcaagcacctccggcccctcaggcaccgcctcaacctcaagcagcccctccacaacaacctgccgctccgcctcaacagcctcaagcccatacactgacaaccctgtctagtgaacctggacagtctcagcgtacccacattaaaaccgagcagctgtcaccgtcacattatagcgaacagcaacagcatagccctcagcaaattgcctattccccgttcaatctgccacactattcaccatcgtatccgccgattactcgtagtcagtatgactataccgatcaccagaacagttcctcgtattatagccatgccgccggtcagggtacaggactgtatagcaccttcacatatatgaatccggcacaacgtccgatgtataccccgattgccgatactagtggagttccgagcattcctcagacccatagccctcaacattgggaacagccggtctatacccaactgacacgccctgggggttatccgtatgatgtcccagattatgccgggggtaaaaaaaaacgtaaagtgggcgggtatggtcgtaaaaaacgccgtcaacgccgccgtggtggccatcatcaccaccatcatggaggaggcctgtttggcgctattgccggctttattgagaatgggtgggaaggcatgattgatggctaa

SEQ ID NO: 8 - DNA, 55 bp, Artificial Sequence
  Primer
agaggaaggtgggcggctatggccgcaaaaaacgccgccagcgccgccgcggcgg

SEQ ID NO: 9 - DNA, 55 bp, Artificial Sequence
  Primer
caatcgcgccaaacaggccgccgccatgatgatgatgatgatggccgccgcggcg

SEQ ID NO: 10 - DNA, 55 bp, Artificial Sequence
  Primer
cacctatccgtatgatgtgccggattatgcgggcggcaagaagaagaggaaggtg

SEQ ID NO: 11 - DNA, 55 bp, Artificial Sequence
  Primer
ttagccatcaatcatgccttcccagccgttttcaataaagcccgcaatcgcgcca

SEQ ID NO: 12 - DNA, 27 bp, Artificial Sequence
  Primer
ggcggctatccgtatgatgtgccggat

SEQ ID NO: 13 - DNA, 24 bp, Artificial Sequence
  Primer
caccatggcgggacacctggcttc

SEQ ID NO: 14 - DNA, 44 bp, Artificial Sequence
  Primer
cggcacatcatacggatagccgccgtttgaatgcatgggagagc

SEQ ID NO: 15 - DNA, 24 bp, Artificial Sequence
  Primer
caccatgagtgtggatccagcttg

SEQ ID NO: 16 - DNA, 44 bp, Artificial Sequence
  Primer
cggcacatcatacggatagccgcccacgtcttcaggttgcatgt

SEQ ID NO: 17 - DNA, 26 bp, Artificial Sequence
  Primer
cttctgcagagaagagtgtcgcaaaa

SEQ ID NO: 18 - DNA, 26 bp, Artificial Sequence
  Primer
ttttgcgacactcttctctgcagaag

SEQ ID NO: 19 - DNA, 26 bp, Artificial Sequence
  Primer
gtcctgcatgcagttccagccaaatt

SEQ ID NO: 20 - DNA, 26 bp, Artificial Sequence
  Primer
aatttggctggaactgcatgcaggac

SEQ ID NO: 21 - DNA, 26 bp, Artificial Sequence
  Primer
caccatgtataatatgatggaaaccg

SEQ ID NO: 22 - DNA, 20 bp, Artificial Sequence
  Primer
ttagccatcaatcatgcctt

SEQ ID NO: 23 - DNA, 21 bp, Artificial Sequence
  Primer
caccatgtctagccctgatgc

SEQ ID NO: 24 - DNA, 20 bp, Artificial Sequence
  Primer
ttagccatcaatcatgcctt

SEQ ID NO: 25 - DNA, 24 bp, Artificial Sequence
  Primer
caccatgcgactctccaaaaccct

SEQ ID NO: 26 - DNA, 44 bp, Artificial Sequence
  Primer
cggcacatcatacggatagccgccgataacttcctgcttggtga

SEQ ID NO: 27 - DNA, 24 bp, Artificial Sequence
  Primer
caccatgtatcagagcttggccat

SEQ ID NO: 28 - DNA, 44 bp, Artificial Sequence
  Primer
cggcacatcatacggatagccgcccgcagtgattatgtccccgt

SEQ ID NO: 29 - DNA, 24 bp, Artificial Sequence
  Primer
caccatgcagtacccgcaccccgg

SEQ ID NO: 30 - DNA, 44 bp, Artificial Sequence
  Primer
cggcacatcatacggatagccgcctccagcattaaaatagcttt

SEQ ID NO: 31 - DNA, 24 bp, Artificial Sequence
  Primer
caccatgcaacgcctggtggcctg

SEQ ID NO: 32 - DNA, 44 bp, Artificial Sequence
  Primer
cggcacatcatacggatagccgccgcagtggccggaggaggcga

SEQ ID NO: 33 - DNA, 24 bp, Artificial Sequence
  Primer
caccatggagctactgtcgccacc

SEQ ID NO: 34 - DNA, 44 bp, Artificial Sequence
  Primer
cggcacatcatacggatagccgccgagcacctggtatatcgggt

SEQ ID NO: 35 - DNA, 24 bp, Artificial Sequence
  Primer
caccatggcatcaaacagcctctt

SEQ ID NO: 36 - DNA, 44 bp, Artificial Sequence
  Primer
cggcacatcatacggatagccgccatatggtcgccaaacagatt

SEQ ID NO: 37 - DNA, 47 bp, Artificial Sequence
  Primer
acaccatgtcagcaaaacttcttttgggatccgagcaccagccggcg

SEQ ID NO: 38 - DNA, 50 bp, Artificial Sequence
  Primer
atggcatcaaacagcctcttcagcacagtgacaccatgtcagcaaaactt

SEQ ID NO: 39 - DNA, 21 bp, Artificial Sequence
  Primer
caccatgaacctggacccttt

SEQ ID NO: 40 - DNA, 20 bp, Artificial Sequence
  Primer
ttagccatcaatcatgcctt

SEQ ID NO: 41 - DNA, 24 bp, Artificial Sequence
  Primer
caccatgagcgccgcgctcttcag

SEQ ID NO: 42 - DNA, 44 bp, Artificial Sequence
  Primer
cggcacatcatacggatagccgccccggcagtctgctgtcccgg

SEQ ID NO: 43 - DNA, 24 bp, Artificial Sequence
  Primer
ccgccgataggcatactacacggc

SEQ ID NO: 44 - DNA, 24 bp, Artificial Sequence
  Primer
ggcggctatccgtatgatgtgccg